(19) United States
(12) Patent Application Publication
(10) Pub. No.: US 2009/0125011 A1
(43) Pub. Date: May 14, 2009

(54) Devices, Methods and Kits for Substantial and Uniform Ablation about a Linear Bipolar Array of Electrodes

(76) Inventor: KAMRAN BEHZADIAN, SUNNYVALE, CA (US)

Correspondence Address: WILSON SONSINI GOODRICH & ROSATI, 650 PAGE MILL ROAD, PALO ALTO, CA, 94304-1050, US

(21) Appl. No.: 12/269,644

(22) Filed: Nov. 12, 2008

Related U.S. Application Data
(63) Continuation-in-part of application No. 11/286,832, filed on Nov. 23, 2005.
Application No. 11/286,832 is a continuation-in-part of application No. 10/878,168, filed on Jun. 28, 2004, now abandoned.

(51) Int. Cl. A61B 18/18

(52) U.S. Cl. 606/33

(57) ABSTRACT

The present invention relates to a medical device for substantially sized and uniform ablation of animal or human tissue comprising a bipolar generator for generating radio frequency at an electrode, a polarity alternator, an applicator probe having a handle and an elongated member, and a tip at the far end of the distal end suitable for insertion into tissue. The elongated member has a proximal and distal end with an electrode cluster of three or more electrodes located on the distal end of the elongated member. The electrodes are electrically insulated from each other and at least two of the electrodes have dissimilar polarity from each other wherein at least one of the electrodes has a high voltage polarity and at least one of the electrodes has a return polarity. During ablation, the polarity alternator automatically and repeatedly changes the polarity of the electrodes to cycle lesion formation along the length of the electrode cluster and alternate lesion formation repeatedly from one polarity to other polarity to form a spherical or near spherical lesion at the distal tip equal in diameter to the length of the cluster.

### CROSS-REFERENCE

This application is a continuation-in-part application of Ser. No. 11/286,832, filed Nov. 23, 2005, which is a continuation-in-part of application Ser. No. 10/878,168, filed Jun. 28, 2004, now abandoned, which are incorporated herein by reference in their entirety and to which applications priority is claimed under 35 U.S.C. § 120.

### FIELD OF THE INVENTION

The invention relates generally to a method and apparatus for utilizing energy, such as radio frequency energy, in a multi electrode and bipolar fashion to treat defined substantial volumes of animal or human tissue uniformly about a single shaft linear bipolar array of electrodes, and particularly have the ability to concentrate lesion formation around desired electrodes through the use of a member having multiple electrodes whose polarity of one or more electrodes is independently controlled and dynamically changed along the length of the electrodes, more particularly in a manner to alternate lesion formation between high voltage and return polarities and is repeated as many times as necessary to produce a large, substantial and uniform lesion.

### BACKGROUND OF THE INVENTION

Methods for treating damaged animal or human tissue, such as those with benign and malignant tumors, have been developed and improved for many years. Recently, a new technique known as radio frequency ablation has been developed in order to treat damaged tissue by destroying its damaged cells plus the adjacent undamaged cells to prevent further spreading. The radio frequency energy causes the tissue to heat up to a high temperature, therefore breaking apart and killing the cells. The objective of radio frequency ablation is to heat tissues to 50-100 degrees centigrade for 4-6 minutes without causing charring or vaporization. Under these conditions there is almost instantaneous cellular protein denaturation, melting of lipid bilayers and destruction of DNA, RNA and key cellular enzymes. This is commonly known as cell necrosis.

In a monopolar ablation system, an applicator probe or catheter containing electrodes with high voltage polarity that releases electrical energy is placed inside the body while an electrode pad with return polarity that completes the electrical circuit is placed outside on the patient's skin. The result is greater amounts of electrical energy being dispersed throughout the patient's body, therefore the possibility of causing collateral damage, or destroying tissue that is not targeted.

Following the development of this device is a new system known as a bipolar ablation system. This improved device contains both the electrodes with high voltage and return polarity on the same applicator that is placed inside the tissue, therefore eliminating the need for an external return electrode pad and eliminates the possibility of collateral damage. However, bipolar instruments available in the market are limited in lesion size and uniformity, or are not suitable for generating a substantial and uniform lesion in solid tissue, or are difficult to use.

Monopolar radiofrequency ablation instruments for substantial ablation in solid tissue marketed by Tyco, Boston Scientific, RITA Medical and others produce spherical or near spherical lesions of 2 to 5 cm in diameter, delivered around an introducer shaft of 1.25 to 2.5 mm diameter with over 100 joules of electrical energy. These monopolar instruments require dispersion of electric energy throughout the body and cause collateral damage such as burns around the return electrode pads, commonly known as pad-burns. Further, products such as those marketed by RITA Medical and Boston Scientific rely on deployment of multiple fragile electrodes in targeted tissue that do not deploy reliably in all tissue and therefore are hard to use.

In bipolar ablation systems, the electric energy is present to the ablated area and not dispersed throughout the body, limiting collateral damage to undesired tissue, but their use is still limited due to limitations in lesion geometry and size.

U.S. Pat. Nos. 6,312,428, 6,524,308 and 6,706,039 describe devices for electro thermal cauterization of tissue with linear electrodes. When operated in a bipolar mode, these devices are limited to spherical lesions of 2 cm or smaller, see FIG. 5. As the inter electrode spacing is increased or as pairs of electrodes are added in series in an attempt to increase the lesion size in these arts, the lesion becomes elliptical or becomes two lobes in solid tissue. Therefore, cauterization zones, or electro thermal lesions made per the arts, are limited in size to fewer than 2 cm when spherical or near spherical in geometry and not comparable to monopolar devices for solid tissue in the market.

U.S. Pat. No. 6,447,506 describes a device for creating long, thin lesions. When operated in a bipolar mode, this device produces long and thin lesions that are about twice as wide as the body width and as long as the electrode cluster. Typical embodiments produce a lesion of 5 cm by 2.5-3 mm wide.

US patent applications 2003/0216727A1 describes a device suitable for body lumens such as the esophagus. Dynamic alteration of polarities of different electrodes is shown in Table 1 to Table 3 of the Long publication, and discussed in paragraphs [0083] to [0086]. This art does not intentionally alternate lesion formation between high voltage and return polarities and thereby limited to body lumens. This art may produce a thin long lesion about a linear bipolar array of electrodes in a body lumen only, and not suitable for solid tumor or tissue. Application of this art as described in Table 1 to solid tumor or tissue would be similar to U.S. Pat. No. 6,524,308 and limited in width to narrow lesions of not wider than 15 mm as shown in FIG. 10 of the U.S. patent.

U.S. Patent applications 2002/0072742A1 and 2002/0022864A1 discuss multi electrodes that are deployed upon placement of a single shaft. These arts are not a single shaft linear bipolar array of electrodes and therefore are hard to use due to difficulty of placement of the device in a targeted tissue, similar to products currently marketed by RITA Medical and Boston Scientific. Further, neither application 2002/0072742A1 nor 2002/0022864A1 manipulate current density in order to alternate lesion formation between high voltage and return polarities.

While technology has resulted in our ability to ablate targeted volumes of tissue, there is still an important need for a bipolar ablation system that heats a zone about a linear cluster of electrodes on a single shaft uniformly, and produces a substantially sized uniform lesion that is nearly spherical and similar in size and uniformity or larger than lesions produced by currently available single needle or multi needle monopolar radiofrequency ablation instruments in solid tissue.

An improved bipolar single needle device for substantial uniform ablation would have to produce uniform lesions that are nearly spherical in geometry, with a diameter of at least 2 to 5 cm around a single shaft similar to monopolar radiofrequency ablation systems available in the market.

The present invention provides such methods that will in turn enable practitioners to ablate a significantly greater amount of targeted areas of tissue, uniformly about a single needle bipolar linear array of electrodes combined with avoiding the unnecessary destruction of other healthy tissue.

### SUMMARY OF THE INVENTION

In a first aspect, the present invention relates to a medical device for substantial and uniform ablation of animal or human tissue comprising of a generator for producing radio frequency electric energy at an electrode, a polarity alternator for dynamically and automatically altering the polarity of individual electrodes in a linear cluster of electrodes and alternate lesion formation between polarities, an applicator probe having a handle, a tip tapered to provide a sharp point, and an elongated member. The elongated member has a proximal end and a distal end. At the distal end, there is a linear electrode cluster of three or more electrodes wherein the electrodes are electrically insulated from each other and at least two of the electrodes have dissimilar polarity from each other. At least one of the electrodes has a high voltage polarity and at least one of the electrodes has a return polarity.

In another aspect of the invention, a polarity alternator is provided for dynamically and semi-automatically altering the polarity of individual electrodes in a linear cluster of electrodes in response to a sensed parameter of a mammalian tissue.

During ablation, polarity alternator may be used for dynamically and automatically changing the polarity of the electrodes in a linear cluster individually at the tip at the far end of the distal end that is suitable for insertion into tissue in a manner to automatically, or semi-automatically, alternate lesion formation between high voltage and return polarities. The electrodes are optionally spaced evenly or optionally unevenly along the length of the distal end of the elongated single shaft member.

The present invention may comprise a tapered distal end fitted with a smaller sharp needle tip. The needle tip allows insertion of the applicator into tissue without an initial cut on the skin.

Optionally, the device is fitted with a conduit for delivery of a suitable material or fluid, such as a therapeutic fluid or saline, to the site of lesion formation.

The electrodes are optionally composed of electrically conductive matter including copper, stainless steel, or precious metal plated material. The electrodes are optionally composed of electrically non-conductive matter such as plastic material plated or painted with a layer of conductive precious metal.

In a second aspect, the present invention relates to a method for substantially uniform ablation of animal or human tissue further comprising the steps of placement of electrode cluster of three or more electrodes on the distal end of an elongated single shaft member adjacent to the tissue. The electrodes are capable of carrying an electrical charge.

Optionally, the method activates a generator with high voltage and ground return polarities. Optionally, a polarity alternator that is located inside the generator, inside the applicator probe handle, or a stand-alone instrument is activated to create dissimilar polarities between the three or more electrodes such that the total surface area of the electrodes with one polarity is unequal to the total surface area of the electrodes having the other polarity, creating a higher current density about the electrodes of polarity with a lesser total surface area sufficient to cause ablation of tissue adjacent to the area of higher current density.

Optionally, the polarity of the three or more electrodes is altered individually to create a higher current density about the electrodes or polarity having a lesser surface area at a different point along the length of the cluster of electrodes prior to altering the polarity of electrodes, to ablate tissue adjacent to the area of higher current density.

The alteration of the polarity of the three or more electrodes will optionally change individually from high voltage to return polarity, neutral, or remain at high voltage polarity and from return to high voltage polarity, neutral, or remain at return polarity.

As an option, the polarity alteration of the three or more electrodes is automatically or semi-automatically repeated during ablation and lesion formation is cycled over the length of the cluster and from electrode to electrode, and lesion formation is altered and tossed between high voltage and ground polarities during each cycle and as many times as needed to achieve a spherical and uniform lesion of the tissue, that is similar or larger in size and uniformity to lesions made by monopolar or bipolar instruments.

In a third aspect, the present invention relates to a medical device adapted and configured to result in uniform, or substantially uniform, ablation of animal or human tissue, the device comprising:
a generator adapted and configured to generate a radio frequency energy;
an applicator probe comprising a cluster of at least three electrodes; and
a polarity alternator operatively connected to the generator and the at least three electrodes, the polarity alternator being adapted to automatically or semi-automatically change polarity of at least one of the electrodes during ablation as to form lesions about at least two electrodes, the polarity alternator further being adapted to cycle lesion formation over a length of the electrode cluster and alternate lesion formation between high voltage and return polarities during each ablation cycle as to create a substantial and uniform ablation about at least two electrodes. This formation step can be performed repeatedly.

The applicator probe may comprise a handle and an elongated member supporting the cluster of at least three electrodes. The elongated member may comprise a proximal and a distal end. In order to avoid electrical short circuiting, the electrodes may be electrically insulated from each other. In one embodiment of the present invention, at least three electrodes are supported by a substantially linear elongated member section.

The generator may be adapted to generate radio frequency energy in the frequency range 200 kHz-1.2 MHz.

The medical device may further comprise a tip at the far end of the distal end, the tip being adapted and configured to penetrate into animal or human tissue.

The at least three electrodes may be spaced evenly along a length of the distal end of the elongated member. Alternatively, the at least three electrodes may be spaced unevenly along a length of the distal end of the elongated member. In still other embodiments, some of the at least three electrodes may be spaced evenly while others of the at least three electrodes are spaced unevenly.

The elongated member may be a substantially rigid member, or alternatively, the elongated member may be flexible, i.e. bendable, or may have a combination of both features varying along its length.

The at least three electrodes may have similar or dissimilar outer surface areas. Additionally, outer surface areas may be adapted and configured to be exposed to a target mammalian tissue, including animal or human tissue.

Each of the at least three electrodes may comprise an electrically conductive material or layer or precious metal-plated material, and each of the at least three electrodes may be shaped in the form of a coil wrapped about a circumference of the elongated member. The coils may be formed by a wire having a substantially rectangular cross-section, and the coils may be wrapped about the circumference of the elongated member allowing flexibility of the electrode cluster. Or alternatively, individual electrodes may be made of machined parts.

In another embodiment the at least three electrodes may be supported by a bendable elongated member section. In another embodiment, the elongated member may be implemented with varying flexibility along its length.

In a fourth aspect, the present invention relates to a method for substantially uniform ablation of animal or human tissue, the method comprising the steps of: placing an applicator probe comprising an electrode cluster of at least three electrodes adjacent to the tissue; applying a dissimilar polarity to the at least three electrodes such that a total surface area of electrodes carrying a first voltage polarity is unequal to a total surface area of an electrode or electrodes carrying a second voltage polarity, thereby creating a higher current density about the electrode or electrodes with the smallest total surface area, thus higher current density being sufficient to cause ablation of tissue; automatically or semi-automatically changing the polarity of at least one of the electrodes so as to spatially shift the higher current density from a first electrode to a second electrode; and changing polarity of at least one of the electrodes during ablation as to alternate and toss lesion formation between high voltage and return polarities to create a substantial and uniform ablation about at least two electrodes. Polarity can be changed repeatedly.

The changing of the polarity of at least one of the electrodes may involve changing from a positive voltage polarity to a neutral or negative voltage polarity. Alternatively, the changing of the polarity of at least one of the electrodes may involve changing from a neutral voltage polarity to a positive or negative voltage polarity. Alternatively, changing of the polarity of at least one of the electrodes may involve changing from a negative voltage polarity to a neutral or positive voltage polarity.

A suitable time period to ablate solid tissue exposed to the higher current density may be within the range of 3 to 10 seconds where lesion is formed about either polarity. In case of a 3 electrode apparatus operating within the stated time range, each lesion cycle over the length of the 3 electrode linear array of electrodes would range from 9 to 30 seconds and lesion would form about high voltage or return polarity for a part of the cycle and lesion would form about the other polarity for the remainder of the cycle. Electrodes and methodology described here may be arranged thus lesion formation time period about each polarity is equal or unequal. Alternatively, a suitable time period to ablate tissue exposed to the higher current density is within the range 0.5 to 3 seconds.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The present invention will now be explained in further details with reference to the accompanying figures, wherein

FIG. 1 illustrates a side block view of a bipolar radio frequency ablation system comprising a generator, a polarity alternator and an applicator probe,

FIGS. 2A and 2B illustrate a bipolar applicator probe with an electrode cluster at the distal tip detailing one possible polarity alternator operation,

FIGS. 3A, 3B, and 3C illustrate side views of a three electrode clusters with individual electrodes energized with different polarities and resulting electric field and lesion concentration zones about high voltage or return polarities,

FIGS. 4A and 4B illustrate an embodiment with flexible coils with rectangular cross section coils with straight edges and a cut away view respectively,

FIG. 5 illustrates a small bipolar lesion made per current according to current conventions,

FIG. 6 illustrates a substantial and uniform lesion resulting from devices made according to current conventions,

FIG. 7 illustrates an embodiment of the device of the invention with three electrodes in the cluster of electrodes.

FIG. 8 illustrates uniform nature of lesion formation in a protein medium,

FIGS. 9A and 9B illustrates lesion formation in muscle and liver tissue respectively,

FIG. 10 illustrates an applicator fitted with a conduit for delivery of fluids to the site of lesion formation ending in a sharp needle tip at the distal end of the applicator.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

REFERENCED PATENTS AND APPLICATIONS

| | |
|---|---|
| US-2003/0216727 A1 | by Long, Gary L. |
| US-2002/0072742 A1 | by Schaefer et al. |
| US-2002/0022864 A1 | by Mahvi et al. |
| US-6,524,308 B1 | by Muller et al. |
| U.S. Pat. No. 6,312,428 | by Eggers et al. |
| U.S. Pat. No. 6,447,506 | by Swanson et al. |

### DETAILED DESCRIPTION OF THE INVENTION

This invention relates to a bipolar radio frequency ablation system and method. Radio frequency ablation may be performed through an open incision or through laparoscopy, which is performed through multiple small incisions, or percutaneously as required. The duration and power requirements of a radio frequency ablation procedure may depend on many factors, including the size of the needed lesion, number of desired applications and location of the animal or human tissue being treated.

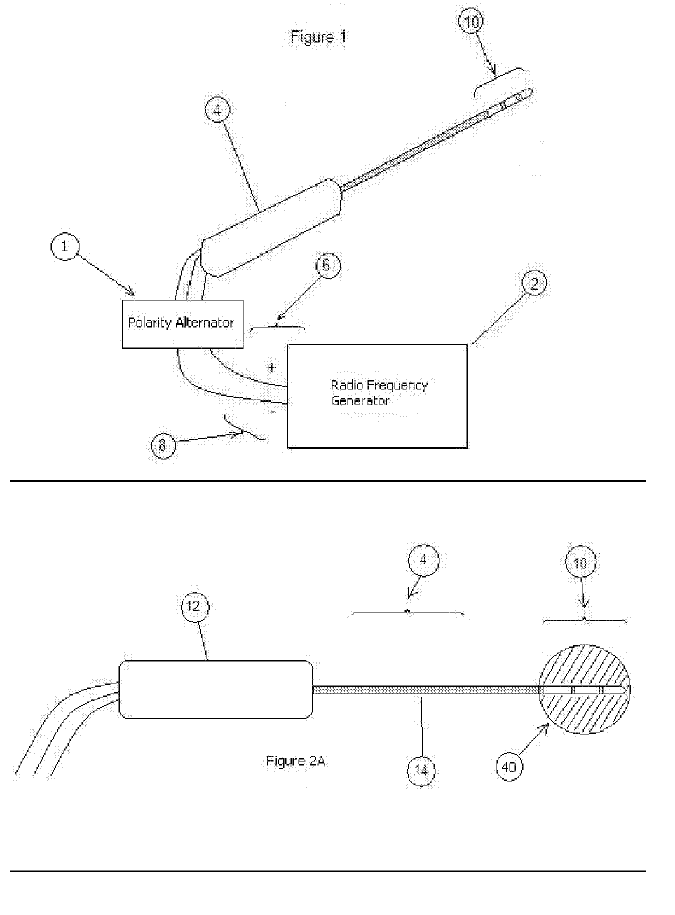

FIG. 1 shows a bipolar ablation system of the invention comprised of a bipolar generator 2, a polarity alternator 1, and a single needle bipolar applicator probe 4 with an electrode cluster that consists of three or more electrodes at the distal tip 10. The probe can be configured such that it forms a slender surgical instrument adapted and configured for exploring a depth or direction of mammalian tissue, a wound, or the like. The bipolar generator 2 may be a conventional general purpose electrosurgical power supply operating at a frequency in the range from about 200 kHz to about 1.2 MHz, with a conventional sinusoidal or non-sinusoidal wave form. The bipolar generator 2 has a positively charged 6 high voltage polarity and a negatively charged 8 return polarity. Such power supplies are available from many commercial suppliers and control power output based on; temperature, current, voltage, or impedance feedback from the applicator or on an activation time basis. The polarity alternator 1 allocates electrical connection to individual electrodes in the cluster 10 and is capable of automatically or semi-automatically altering individual electrodes from high voltage polarity to return polarity or neutral and from return polarity to high voltage polarity or neutral and dynamically repeating the polarity alterations during ablation.

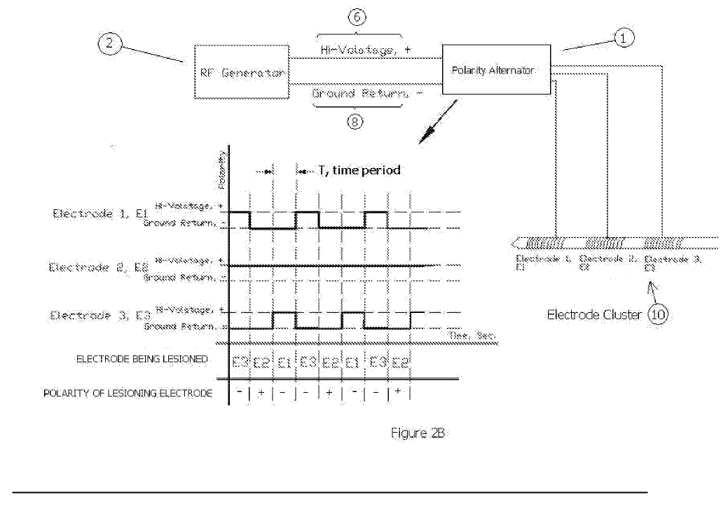

The applicator probe 4, as shown in FIG. 2A is comprised of a handle 12, an elongated member 14, and a linear electrode cluster with three or more electrodes 10. Formation of a substantial and uniform lesion 40 is shown around the linear cluster 10.

FIG. 2B shows only one possible method of polarity alteration and alteration of lesion formation about two polarities during each cycle of lesion formation. In the example method shown, E2 is adapted to remain at high voltage polarity while polarity of other electrodes alters.

Polarity alternator 1 alters the individual polarities to the electrode array during ablation hence that lesion is initially formed about electrode E3 then electrode E2 then electrode E1 for a preset time period T about each electrode of andy polarity, and then lesion formation can be repeated about electrodes E3, E2, E1 and then back to E3 so on. As such, lesion formation repeating about electrodes E3, E2, and E1 and then back to E3 are referred to as cycle of lesion formation and equal to 3 times T.

FIG. 2B shows high voltage and return polarities of a radiofrequency generator into the polarity alternator. T is the time period lesion is formed about each electrode of either polarity. Polarity alternator 1 changes polarity of individual input to a cluster of three electrodes as shown in FIG. 2B. During each cycle of lesion formation equal to 3 times T, for a 3 electrode cluster over the length of the three electrode applicator probe shown in FIG. 2B, the lesion is selectively formed about high voltage polarity and then altered to form about return polarity. In this particular representation of devices known by those skilled in the art, lesion is formed about high voltage polarity one third of the cycle and lesion is formed about ground polarity two third of the cycle time.

The ability to alternate or “toss” lesion formation between high voltage and ground polarities, as shown in FIG. 2B and denoted as “Electrode being Lesioned” and “Polarity of Lesioning Electrode” allows the lesion to become “Super-Sized” and grow to a nearly spherical shape of substantial size in solid tissue not duplicated by previously known methods and devices.

Polarity alternator 1 is shown as a stand-alone instrument in FIG. 2B, however it may optionally be located inside the generator 2 or inside handle 12.

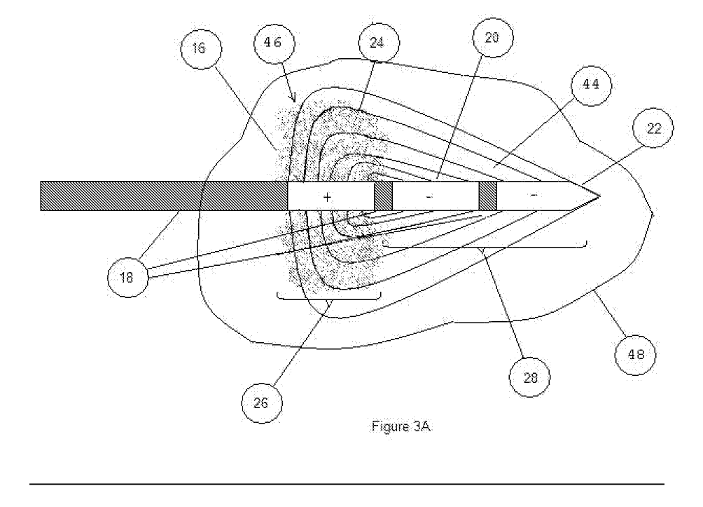

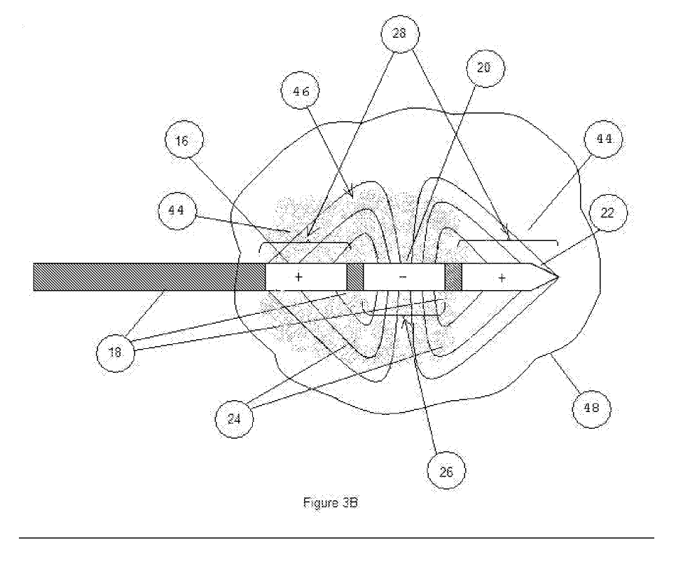

FIGS. 3A through 3C are examples of steps of ablating tissue using a bipolar applicator 4 with an electrode cluster that consists of three or more electrodes at the distal tip 10 and with a resulting electric field when electrodes are identical in composition and geometry. Alteration of polarity is repeated to cycle lesion formation about individual electrodes and alternate lesion formation between high voltage and return polarities during ablation and can ultimately result in a substantially sized spherical lesion.

FIG. 3A shows dissimilar polarity by electrode 16 being energized with high voltage polarity, 6 which is denoted by a “+” sign, and electrodes 20 and 22 being energized with return polarity 8, which is denoted by a “−” sign. Items 18 are electrical insulation and can be found between the electrodes and on the proximal end of the applicator. Dissimilar surface areas, shown with smaller surface area 26 and larger surface area 28, allows the electric field and therefore electrical current density to be higher 24 about high voltage electrode 16 and lower 44 about return polarity electrodes 20 and 22, therefore producing a higher current density 24 and lower current density 44, thus ablation heat generation is greatest about electrode 16 resulting in lesion generation 46 in targeted tissue 48 about electrode 16 that has a + polarity and for a time period, T. The electrodes may be spaced evenly or unevenly with respect to electrical insulation 18 found in between each electrode.

Next step is represented in FIG. 3B. FIG. 3B shows dissimilar polarity by electrode 20 being energized with return polarity, − and electrodes 16 and 22 being energized with high voltage polarity, +. Item 18 are electrical insulation. Dissimilar surface areas allow the electric field and therefore electric current density to be higher 24 about electrode 20 having a return polarity and lower 44 about electrodes 16 and 22 having high voltage polarity, therefore producing a higher current density 24 and lower current density 44, thus ablation heat generation is greatest about electrode 20 resulting in lesion generation 46 in targeted solid tissue 48 about electrode 20 which has a − polarity and for a time period T. The electrodes may be spaced evenly or unevenly with respect to electrical insulation 18 found in between each electrode. It should be noted that lesion formation has altered from a + polarity electrode to a − polarity electrode, as shown in the figure.

Next step is represented in FIG. 3C. FIG. 3C shows dissimilar polarity by electrode 22 being energized with return polarity, − and electrodes 16 and 20 being energized with high voltage polarity, +. Item 18 are electrical insulation. Dissimilar surface areas allow the electric field and therefore electric current density to be higher 24 about electrode 22 having − polarity and lower 44 about electrodes 16 and 20 or + polarity, therefore producing a higher current density 24 and lower current density 44, thus ablation heat generation is greatest about electrode 22 resulting in lesion generation 46 in targeted tissue 48 about electrode 22 which is has a − polarity for a time period T. The electrodes may be spaced evenly or unevenly with respect to electrical insulation 18 found in between each electrode. It should be noted that, lesion formation about electrode 26 is of − polarity and can change back to lesion formation about + polarity in the next repetition of lesion formation cycle.

Lesion formation can be independent of polarity because it can form where a higher current density is present. Additionally, current density is independent of polarity and a function of active surface areas. One or more lesions form about the electrode, or electrodes with the higher current density, irrespective of a particular polarity. Therefore, electrodes with high voltage polarity or return polarity in FIGS. 3A through 3C may have their polarities altered in order to shift high current density to the tissue surrounding a different electrode polarity. The ability to alternate lesion formation between high voltage and return polarities allows the lesion to grow to a large and substantial size.

The polarity alteration can be dynamically repeated to cycle lesion formation through the three electrodes as shown in FIGS. 3A, 3B and 3C during ablation as shown in FIG. 2B. Lesion may initially form as a long and thin volume about the linear array of electrodes during the first few cycles of lesion formation similar to references known in the art, but lesion eventually becomes a near sphere upon repeated cycling of lesion formation and alteration of lesion formation between high voltage and return polarities about individual electrodes over the length of the array. The spherical or near spherical lesion that is formed has a diameter equal to the length of the electrode cluster and is uniform.

In one embodiment, a cluster 10 of three 12 mm long electrodes separated by 5 mm length of insulation each on a 1.75 mm shaft 14 with a lesion formation time period T of 5 to 10 seconds about each electrode in the cycle may produce a spherical or near spherical lesion of diameter 45 to 50 mm with 12 to 15 watts of input radiofrequency power in about 15 minutes of ablation time.

In another embodiment, a cluster 10 of three 3 mm long electrodes separated by 1 mm length of insulation each on a 0.5 mm shaft 14 with a lesion formation time period T of 2 to 3 seconds about each electrode in the cycle can produce a spherical or near spherical lesion of diameter 10 to 12 mm with about 2 to 5 watts of input radiofrequency power in about 4 minutes of ablation time.

In yet another embodiment, a cluster 10 of three 1 mm long electrodes separated by 1 mm length of insulation each on a 0.5 mm shaft 14 with a lesion formation time period T of 0.5 to 1.5 seconds about each electrode in the cycle can produce a spherical or near spherical lesion of diameter of 5 mm with about 1 to 2 watts of input radiofrequency power in about 1 to 2 minutes of ablation time.

It is appreciated that polarity alteration shown in FIG. 2B is one possible method of dynamically repeating lesion formation about a linear array of three electrodes and cycling lesion formation over the length of the electrodes and between high voltage and return polarities during ablation. It is also appreciated that there are many formats for dynamically changing polarities in a linear bipolar array of electrodes to cycle lesion formation over the length of an array of electrodes and alternating lesion formation between high voltage and return polarities in order to produce a substantially sized uniform lesion of spherical or near spherical geometry. The lesion formation cycle can be repeated as often as desired.

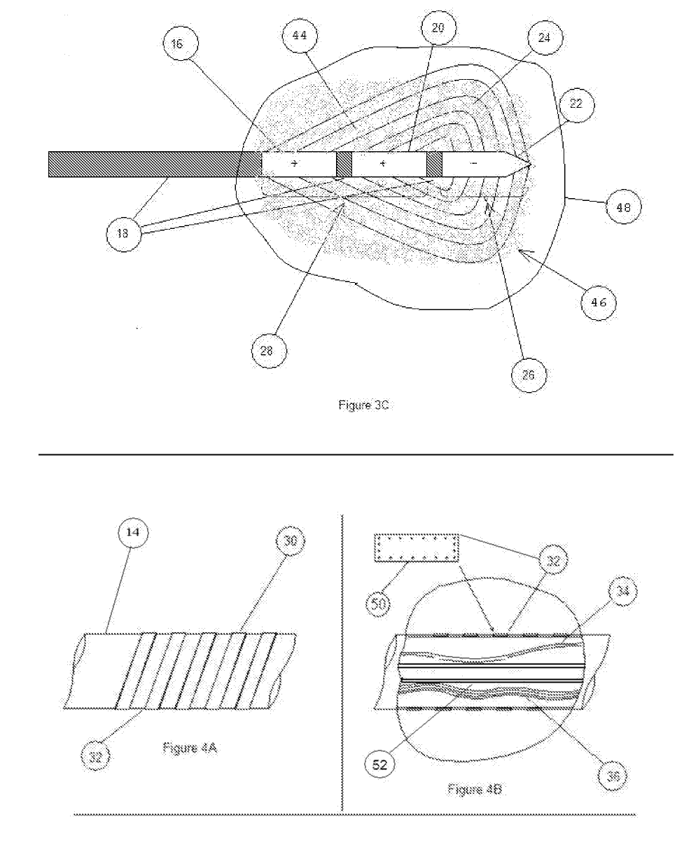

FIG. 4A shows a single electrode made up of flat wire with sharp and straight edges 32 wrapped about the circumference of the elongated member 14 in the form of a flexible coil 30 or a machined part with grooves cut into the outer surface. A higher local current density 24 is present about the straight edges 32.

FIG. 4B shows a rectangular cross section 50 of the flexible coiled electrode 30 with straight edges 32 and temperature feedback thermocouple wires 34 and individual wires to individual electrodes 36. Conduit tube 51 of FIG. 10 is shown in FIG. 4B.

An electrode shaped in the form of flexible coils 30 with straight edges 32 wrapped about the circumference of the elongated member 14 with rectangular cross section 50 can produce more uniform ablations over the length of the electrode during the first few lesion formation cycles in addition to being flexible. Although flexible coils with non-rectangular cross sections such as round may be used to achieve flexibility, a rectangular shape is preferred because an individual electrode made up of many straight edges 32 spaced closely to each other produces a more uniform ablation that is made up of smaller lesions about the straight edges 32 that are spaced closely to each other that propagate and join into a uniform lesion about the entire length of the individual electrode. The power input to the electrode array is controlled by monitoring lesion temperature by locating feedback temperature sensors such as thermocouple wires 34 under electrodes which may allow for automatic or semi-automatic control of the electrode array. Power can then be adjusted to maintain, for example, an average temperature under 100 degrees centigrade around the linear array. As will be appreciated by those skilled in the art, sensors can also be provided that are adapted and configured to sense other tissue parameters in order to provide feedback for the operation of the electrodes, wherein the feedback is acted on automatically by the device to adjust its operational parameters or is acted upon by a user to adjust the device operational parameters.

Determination of lesion formation and completion is accomplished by monitoring electrical current and voltage in individual wire connections 36 to electrodes in circuitry embedded within the generator 2 or polarity alternator 1. Electrical characteristics such as impedance of the tissue under treatment are calculated by the circuitry based on the monitored voltage and current. The circuit continuously monitors and calculates the electrical characteristics of the lesion that is being formed around the electrode where lesion is being formed and the other electrodes and then averages the values over a cycle for all electrodes in a cluster.

Lesion is formed as tissue cell necroses takes place. As cells change composition while the tissue is heated, the average impedance of the volume of tissue where electric current is contained continuously changes, until full necroses of all cells in the volume of tissue where the electric field is present takes place. Lesion grows to the size of the electric field because that is where electrons responsible for ablation are present. As lesion is formed and grows to the size of the electric field, the average impedance of tissue where electric energy is present, continuously changes until full lesion formation. Average impedance measured between electrodes does not change any further; when a lesion size of the electric field about a linear array of electrodes is formed. Substantial and uniform ablations 40 only treat the material portions of tissue that is located in a sphere or near-sphere around the distal tip of the applicator 4 and equal in diameter to the length of the bipolar cluster 10 as shown in FIG. 2A.

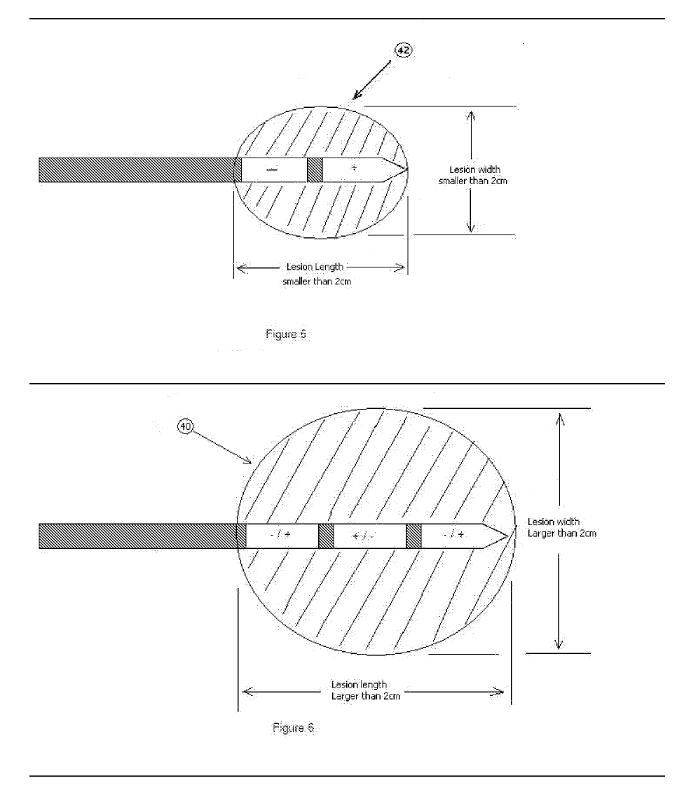

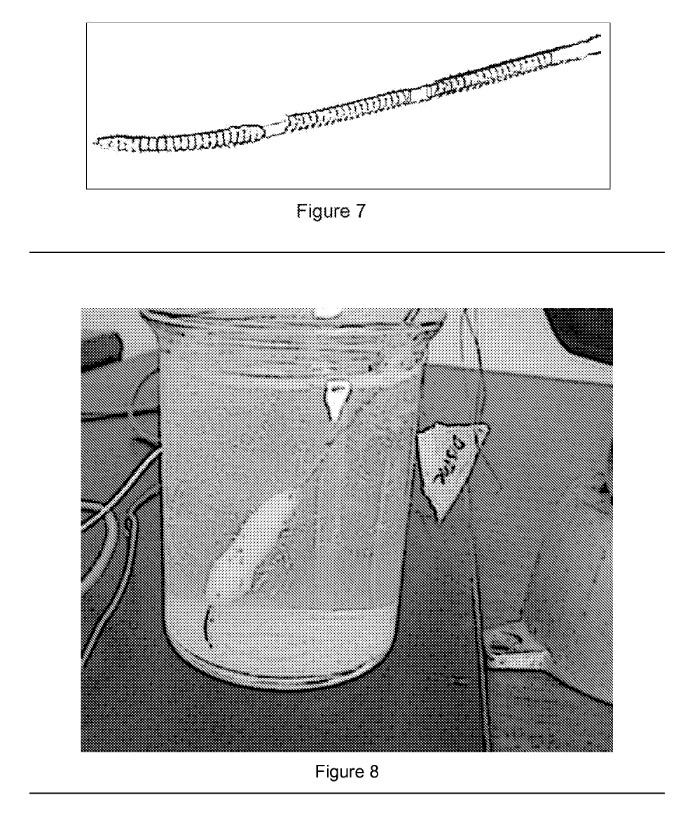

FIG. 5 shows a spherical or near spherical lesion that is possible with, for example, U.S. Pat. Nos. 6,312,428; 6,524,308 and 6,706,039. Spherical or near spherical lesions made by prior art are under 2 cm in width or length. Addition of electrode pairs, increasing the length of the electrodes, or increasing the space between electrodes does not affect lesion width and can only increase lesion length.

FIG. 6 shows a uniformly heated zone around a bipolar array of electrodes made per disclosed art. Uniform and substantial lesions are spherical or near spherical with lesion widths equal or near-equal to lesion length and therefore spherical or near-spherical with diameters ranging from 5 mm to 10 cm or larger depending on applicator electrode cluster length.

The ability to control lesion formation about specific electrodes in a bipolar device and cycle lesion formation over the length of a cluster of electrodes repeatedly and alter lesion formation between ground and high voltage polarities by alteration of individual electrode polarities, enables the device to produce a substantial uniform ablation lesion 40 by manipulating current densities about electrodes in a linear array of electrodes and cycling the current density and therefore lesion formation many times over the length of the cluster and altering lesion formation between polarities, resulting in a substantial uniform ablation of the tissue along the length of the electrode cluster that is spherical or near spherical with a diameter about equal to the length of the electrode cluster, as shown in FIG. 6.

FIG. 10 shows an applicator probe and tip with a conduit 51 suitable for irrigation of a lesion site at the distal tip of an applicator with a therapeutic fluid such as saline. A conduit tube 51 with a lure fitting 53 at the proximal end and a sharp needle tip 52 at the distal tip is fitted throughout the length of the applicator and is suitable for irrigation of the lesion site with a fluid.

Normally, a small razor cut is made in the skin before an applicator is inserted into the body. Providing a small sharp needle tip in the center of a symmetric distal tip can allow insertion of an applicator into the body without the need for an initial cut in the skin and can maintain the overall symmetry of an applicator tip. An overall symmetry of an applicator tip facilitates navigation of an applicator probe to a location of interest and can provide the practitioner with ease of use.

### EXAMPLE

Lesion formation per this patent may be verified in many mediums such as a transparent protein medium like an egg white or in animal tissue such as; muscle tissue, liver tissue, kidney tissue, lung tissue, bone, cartilage, etc.

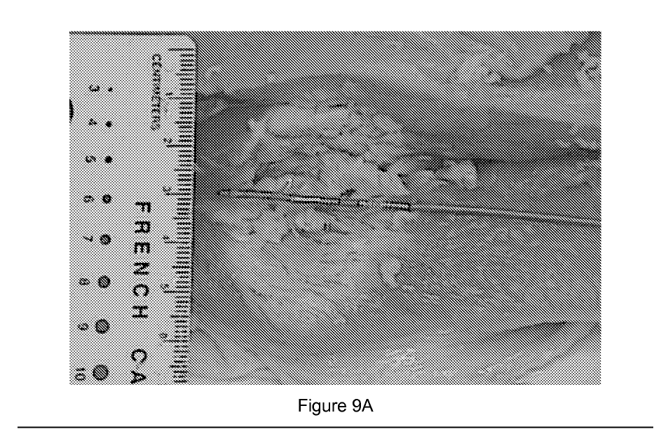

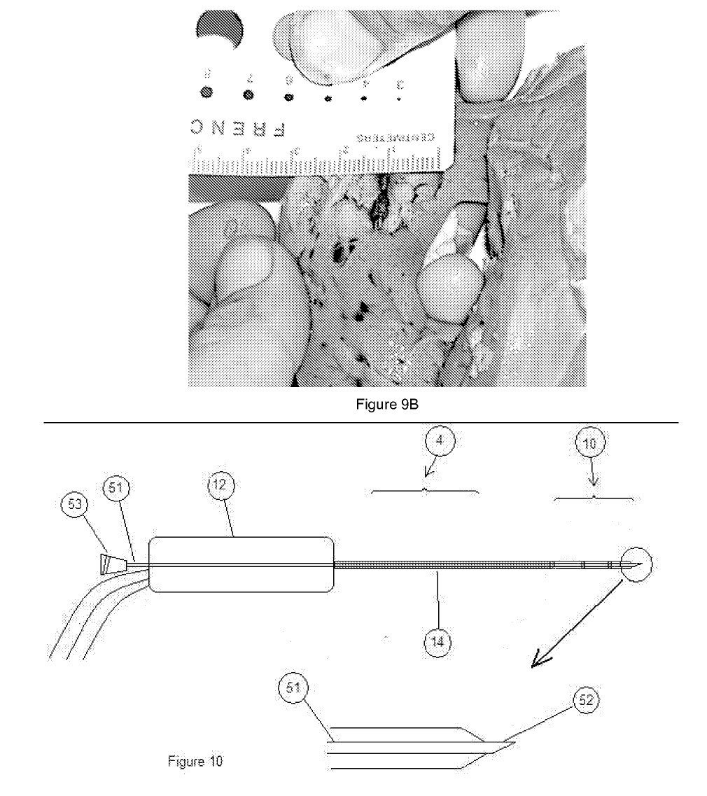

FIG. 9A shows a lesion in muscle tissue formed per this invention. Lesion is produced and then the tissue is sectioned in order to show the uniform and substantial nature of the lesion. Lesion is produced by cycling the lesion formation at T=3 sec per electrode, for a total time of approximately 15 minutes, or about 100 repeated cycles of lesion formation along the length of the electrode array. Lesion formation about each electrode is repeated many times in a cycled fashion from electrode to electrode and altered between polarities along the length of the cluster per the polarity alteration method of FIG. 2B, where a substantial and uniform spherical lesion is produced by repeated cycling of lesion formation about E3, E2, E1, and then back to E3, and so on throughout ablation. A lesion formation cycle over electrodes E3, E2 and E1 in this example is 9 seconds, where lesion is formed about high voltage polarity for 3 seconds and formed about return polarity for 6 seconds during each cycle. Lesion is spherical and has a diameter of approximately 50 mm that is about equal to the length of the three-electrode cluster of 48 mm. In this example electrodes are 13 mm each and are separated by about 5 mm of insulation on a shaft of 1.75 mm. Lesion is formed with about 15 watts of input radiofrequency power.

A lesion in liver tissue is shown in FIG. 9B. Lesion is produced and then tissue is sectioned in order to show the uniform nature of the lesion. Lesion is made around a 20 mm electrode cluster made up of three 5 mm each electrodes, separated by about 2.5 mm length of insulation on a shaft of 1.5 mm with a lesion formation time period of T=2 seconds about each electrode in the cycle. A lesion formation cycle in this example is 6 seconds, where lesion is formed about high voltage polarity for 2 seconds and formed about return polarity for 4 seconds during each cycle. Lesion is made in about 6 minutes of ablation time or 60 repeated cycles of lesion formation about the length of the electrode cluster. Resulting lesion is spherical with a diameter of about 20 mm and equal to the length of the electrode cluster, and it was formed with about 5 watts of radiofrequency power.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A medical device for substantially uniform ablation of animal or human tissue, the device comprising:
a generator for generating radio frequency energy;
an applicator probe comprising a cluster of at least three electrodes; and
a polarity alternator operatively connected to the generator and the at least three electrodes, the polarity alternator being adapted to change polarity of at least one of the electrodes during ablation to form lesions about at least two electrodes, the polarity alternator further being adapted to cycle lesion formation over a length of the electrode cluster during ablation and to alternate lesion formation between polarities.

2. The device of claim 1 wherein the lesion formation cycle is for an equal or unequal time duration.

3. The device of claim 1 wherein a substantial and uniform ablation is performable about at least one electrode.

4. The device of claim 1, wherein the applicator probe comprises a handle and an elongated single shaft member supporting the cluster of at least three electrodes.

5. The device of claim 4, wherein the elongated member comprises a proximal and a distal end.

6. The device of claim 1, wherein the electrodes are electrically insulated from each other.

7. The device of claim 4, wherein the at least three electrodes are supported by a substantially linear single shaft elongated member section.

8. The device of claim 1, wherein the generator is adapted to generate radio frequency energy in the frequency range 200 kHz-1.2 MHz.

9. The device of claim 5, further comprising a tip at the far end of the distal end, the tip being suitable for penetrating into animal or human tissue.

10. The device of claim 5, wherein the at least three electrodes are spaced evenly along a length of the distal end of the elongated member.

11. The device of claim 5, wherein the at least three electrodes are spaced unevenly along a length of the distal end of the elongated member.

12. The device of claim 4, wherein the elongated member is substantially rigid.

13. The device of claim 4, wherein the elongated member is flexible.

14. The device of claim 1, wherein the at least three electrodes have dissimilar outer surface areas, the outer surface areas being adapted to be exposed to mammalian tissue.

15. The device of claim 1, wherein each of the at least three electrodes comprises an electrically conductive matter or precious metal-plated material.

16. The device of claim 4, wherein the at least three electrodes are supported by a bendable elongated member section.

17. The device of claim 4, wherein the elongated member comprises a varying flexibility along its length direction.

18. A method for substantially uniform ablation of mammalian tissue, the method comprising the steps of:
placing an applicator probe comprising an electrode cluster of at least three electrodes adjacent to a tissue;
applying a dissimilar polarity to the at least three electrodes such that a total surface area of electrodes carrying a first voltage polarity is unequal to a total surface area of an electrode or electrodes carrying a second voltage polarity, thereby creating a higher current density about the electrode or electrodes with the smallest total surface area, the higher current density being sufficient to cause ablation of tissue;
changing the polarity of at least one of the electrodes to spatially shift the higher current density from a first electrode to a second electrode; and
changing polarity of at least one of the electrodes during ablation a plurality of times to cycle lesion formation over the length of the electrode cluster during ablation and to alternate lesion formation between the two polarities for equal or unequal time duration during each cycle to create a substantial and uniform ablation about at least two electrodes.

19. The method of claim 18, wherein the changing of the polarity of at least one of the electrodes involves changing from a positive voltage polarity to a neutral or negative voltage polarity.

20. The method of claim 18, wherein the changing of the polarity of at least one of the electrodes involves changing from a neutral voltage polarity to a positive or negative voltage polarity.

21. The method of claim 18, wherein the changing of the polarity of at least one of the electrodes involves changing from a negative voltage polarity to a neutral or positive voltage polarity.

22. The method of claim 18, wherein a time period to ablate tissue exposed to the higher current density of positive or negative polarity is within the range 3 to 10 seconds.

23. The method of claim 18, wherein a time period to ablate tissue exposed to the higher current density of positive or negative polarity is within the range 0.5 to 3 seconds.

24. A kit for ablating a human tissue comprising:
a medical device further comprising a generator for generating radio frequency energy; an applicator probe comprising a cluster of at least three electrodes; and a polarity alternator operatively connected to the generator and the at least three electrodes, the polarity alternator being adapted to change polarity of at least one of the electrodes during ablation to form lesions about at least two electrodes, the polarity alternator further being adapted to cycle lesion formation over a length of the electrode cluster during ablation and to alternate lesion formation between polarities; and
one or more of a scalpel, a probe tip, antiseptic.
